(19) United States
(12) Patent Application Publication
(10) Pub. No.: US 2022/0387474 A1
(43) Pub. Date: Dec. 8, 2022

(54) SUPPLEMENT COMPOSITIONS FOR NITROUS OXIDE PATIENTS

(71) Applicant: Peter Nagele, Chicago, IL (US)

(72) Inventor: Peter Nagele, Chicago, IL (US)

(21) Appl. No.: 17/805,749

(22) Filed: Jun. 7, 2022

Related U.S. Application Data
(60) Provisional application No. 63/202,320, filed on Jun. 7, 2021.

(51) Int. Cl. A61K 31/714; A61K 31/519; A61K 31/4415; A61K 31/198; A61K 31/205; A61K 33/00

(52) U.S. Cl. CPC A61K 31/714; A61K 31/519; A61K 31/4415; A61K 31/198; A61K 31/205; A61K 33/00

(57) ABSTRACT

The present invention generally relates to supplement compositions for patients receiving current or chronic nitrous oxide treatment.

### FIELD OF THE INVENTION

The present invention generally relates to supplement compositions for patients receiving current or chronic nitrous oxide treatment.

### BACKGROUND OF THE INVENTION

WO2015175531 describes how nitrous oxide (laughing gas)(50% by weight, inhaled concentration) improved depressive symptoms in patients with treatment-resistant major depression (TRMD). One thing not mentioned in WO2015175531 is the potential side effects of chronic nitrous oxide exposure. One of the potential side effects is the inactivation of vitamin B₁₂ and subsequently methionine synthase which can cause blood changes and nervous system damage.

Thus, there is a need for compositions and methods of treating nitrous oxide side effects.

### SUMMARY OF THE INVENTION

In an aspect, the present invention provides a novel supplement composition designed to counter the side effects of nitrous oxide treatment.

In another aspect, the present invention provides a novel of treating the side effects of nitrous oxide treatment.

These and other aspects, which will become apparent during the following detailed description, have been achieved by the inventors' discovery of a novel supplement composition for use with treating the side effects of nitrous oxide treatment.

### DETAILED DESCRIPTION OF THE PREFERRED ASPECTS

Exemplary aspects of the present invention are described with reference to the figures, where appropriate. Although the following detailed description contains many specifics for purposes of illustration, a person of ordinary skill in the art will appreciate that variations and alterations to the following details are within the scope of the invention. Accordingly, the following aspects of the invention are set forth without any loss of generality to, and without imposing limitations upon, the claimed invention.

### Abbreviations and Definitions

When introducing elements of the present disclosure or an aspect thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

Nitrous oxide treatment refers to the treatment protocol wherein patients are treated for depression via therapeutic administration of nitrous oxide gas.

Current refers to nitrous oxide treatments that a patient is receiving contemporaneously with administration of a supplement composition described herein.

Chronic refers to nitrous oxide treatments that are continued for at least 2 months.

The terms “treatment,” “treating” or “treat,” when referring to a condition, and as understood in the art, are defined to mean an approach for obtaining beneficial or desired results, including clinical results. Beneficial or desired clinical results can include alleviation of one or more symptoms of the condition, diminishment of extent of disease or condition, stabilized (i.e., not worsening) state of disease or condition, preventing spread of disease, delay or slowing of disease progression, palliation of the disease state, and remission (whether partial or total), whether detectable or undetectable.

The terms “subject” or “patient” are used interchangeably and mean humans who are receiving nitrous oxide (current nitrous oxide treatment)(e.g., as a treatment for depression).

The term “subject in need thereof” means a subject having a condition that can be treated with the present supplement (e.g., a subject receiving current or chronic nitrous oxide treatment and has or likely will have vitamin and/or amino acid deficiencies resulting therefrom).

The term “effective amount” or “pharmaceutically effective amount” are used interchangeably and are defined to mean the amount or quantity of supplement (or individual components thereof), which is (are) sufficient to elicit an appreciable biological response when administered to a patient.

Vitamin B₁₂, also known as cobalamin, is a well-known vitamin. It consists of 4 vitamers, cyanocobalamin, hydroxocobalamin, adenosylcobalamin and methylcobalamin with cyanocobalamin and hydroxocobalamin being converted into adenosylcobalamin and methylcobalamin, the physiologically active vitamers). Thus, Vitamin B₁₂, as used herein refers to a mixture of the 4 vitamers and, alternatively to 1 or a mixture of 2 or 3 of the 4 vitamers. Vitamin B₁₂ also includes B₁₂ precursors including methyl cobalamin.

Folate, also known as vitamin B₉, is a well-known vitamin. Folate is derived from folic acid, which, when administered, is converted to folate by the human body. Folic acid is N-(4-{[(2-amino-4-oxo-1,4-dihydropteridin-6-yl)methyl]amino}benzoyl)-L-glutamic acid. Other folate precursors can be administered in place of folic acid. These other precursors include folinic acid, methylated, methenylated and formylated forms of folates, their salts or esters, as well as their derivatives with one or more glutamic acid, and all in either reduced or oxidized form.

Vitamin B₆, also known as pyridoxine, is a well-known vitamin. It is converted in the human body to the active form of vitamin B₆. Because the active form of vitamin B₆ is made in vivo, other precursors can be administered in place of pyridoxine. These other precursors include pyridoxine-5′-phosphate, pyridoxal, pyridoxal-5′-phosphate, pyridoxamine, pyridoxamine-5′-phosphate, 4-pyridoxic acid, and pyritinol.

L-methionine is 2-amino-4-(methylthio)butanoic acid, an essential amino acid.

Betaine is N,N,N-trimethylglycine (the trimethylated form of amino acid glycine). Both the anhydrous and HCl forms are included.

Methods and Compositions

In an aspect, the present invention provides a novel supplement composition, comprising:
a. a therapeutically effective amount of vitamin B₁₂ (or a precursor thereof);
b. a therapeutically effective amount of folic acid (or another folate precursor);
c. a therapeutically effective amount of vitamin B₆ (or a precursor thereof);
d. a therapeutically effective amount of L-methionine; and,
e. a therapeutically effective amount of Betaine.

In another aspect, the vitamin B₁₂ (or a precursor thereof) is substantially isolated.

In another aspect, the folic acid (or another folate precursor) is substantially isolated.

In another aspect, the vitamin B₆ (or a precursor thereof) is substantially isolated.

In another aspect, the L-methionine is substantially isolated.

In another aspect, the Betaine is substantially isolated.

In another aspect, substantially isolate means that the component, if naturally occurring is substantially isolated from the natural source prior to addition to the supplement composition. In another aspect, examples of substantially isolated include the component being at least 50, 60, 70, 80, 90, 95, to 99% free from materials from its natural source (or synthetic materials if synthetically made) prior to additional to the supplement composition.

In another aspect, examples of substantially isolated include the component being at least 50, 60, 70, 80, 90, 95, to 99% pure exclusive of the other components of the supplement composition.

In another aspect, a therapeutically effective amount of vitamin B₁₂ (or a precursor thereof) is an amount sufficient to measurably increase the vitamin B₁₂ levels of a subject after one or more administrations of a supplement composition of the present invention.

In another aspect, a therapeutically effective amount of folic acid (or another folate precursor) is an amount sufficient to measurably increase the folate levels of a subject after one or more administrations of a supplement composition of the present invention.

In another aspect, a therapeutically effective amount of vitamin B₆ (or a precursor thereof) is an amount sufficient to measurably reduce homocysteine levels of a subject after one or more administrations of a supplement composition of the present invention.

In another aspect, a therapeutically effective amount of L-methionine is an amount sufficient to measurably increase the L-methionine levels of a subject after one or more administrations of a supplement composition of the present invention.

In another aspect, a therapeutically effective amount of Betaine is an amount sufficient to measurably reduce homocysteine levels of a subject after one or more administrations of a supplement composition of the present invention.

Examples of the amount of vitamin B₁₂ (or an equivalent amount of a precursor) in a composition of the present invention include 2, 3, 4, 5, 6, 7, 8, 9, 10, 11, to 12 μg. Additional examples include (a) 5 μg, (b) 6 μg, and (c) 7 μg.

Examples of the amount of folic acid (or an equivalent amount of another folate precursor) in a composition of the present invention include 100, 110, 120, 130, 140, 150, 160, 170, 180, 190, 200, 210, 220, 230, 240, 250, 260, 270, 280, 290, to 300 mg. Additional examples include (a) 150 mg, (b) 200 mg, and (c) 250 mg.

Examples of the amount of vitamin B₆ (or an equivalent amount of a precursor) in a composition of the present invention acid include 0.5, 0.6, 0.7, 0.8, 0.9, 1, 1.2, 1.4, 1.6, 1.8, 2, 2.2, 2.4, 2.6, 2.8, 3, 3.2, 3.4, 3.6, 3.8, to 4 mg. Additional examples include (a) 1 mg, (b) 2 mg, and (c) 3 mg.

Examples of the amount of L-methionine in a composition of the present invention include 200, 210, 220, 230, 240, 250, 260, 270, 280, 290, 300, 310, 320, 330, 340, 350, 360, 370, 380, 390, to 400 mg. Additional examples include (a) 250 mg, (b) 300 mg, and (c) 350 mg.

Examples of the amount of Betaine in a composition of the present invention include 200, 210, 220, 230, 240, 250, 260, 270, 280, 290, 300, 310, 320, 330, 340, 350, 360, 370, 380, 390, to 400 mg. Additional examples include (a) 250 mg, (b) 300 mg, and (c) 350 mg.

In another aspect, the present invention provides a novel supplement composition, comprising:
a. a therapeutically effective amount of vitamin B₁₂;
b. a therapeutically effective amount of folic acid;
c. a therapeutically effective amount of vitamin B₆;
d. a therapeutically effective amount of L-methionine; and,
e. a therapeutically effective amount of Betaine.

In another aspect, the present invention provides a novel supplement composition, comprising:
a. 2-12 mcg of vitamin B₁₂ or a precursor thereof;
b. 100-300 mg folic acid or another folate precursor;
c. 0.5-4 mg of vitamin B₆ or a precursor thereof;
d. 200-400 mg of L-methionine; and,
e. 200-400 mg of Betaine.

In another aspect, the present invention provides a novel supplement composition, comprising:
a. 2-12 mcg of vitamin B₁₂;
b. 100-300 mg folic acid;
c. 0.5-4 mg of vitamin B₆;
d. 200-400 mg of L-methionine; and,
e. 200-400 mg of Betaine.

In another aspect, the present invention provides a novel supplement composition, comprising:
a. 4-8 mcg of vitamin B₁₂;
b. 150-250 mg folic acid;
c. 1-3 mg of vitamin B₆;
d. 250-350 mg of L-methionine; and,
e. 250-350 mg of Betaine.

In another aspect, the present invention provides a novel supplement composition, comprising:
a. 3 mcg of vitamin B₁₂;
b. 200 mg folic acid;
c. 2 mg of vitamin B₆;
d. 300 mg of L-methionine; and,
e. 300 mg of Betaine.

In another aspect, the present invention provides a novel method of treating a subject, comprising: administering a therapeutically effective amount of a composition, comprising:
a. vitamin B₁₂ or a precursor thereof;
b. folic acid or another folate precursor;
c. vitamin B₆ or a precursor thereof;
d. L-methionine; and,
e. Betaine;

wherein the subject is receiving current or chronic nitrous oxide treatment.

In another aspect, the subject who is receiving current or chronic nitrous oxide treatment is known to have at least one of the following conditions:
a. vitamin B₁₂ deficiency;
b. folate deficiency;
c. vitamin B₆ deficiency; and,
d. elevated homocysteine levels.

In another aspect, the composition used in the method of treating, comprises:
a. vitamin B₁₂;
b. folic acid;
c. vitamin B₆;
d. L-methionine; and,
e. Betaine;

In another aspect, the composition used in the method of treating, comprises:
a. 2-12 mcg of vitamin B₁₂ or a precursor thereof;
b. 100-300 mg folic acid or another folate precursor;
c. 0.5-4 mg of vitamin B₆ or an equivalent precursor;
d. 200-400 mg of L-methionine; and,
e. 200-400 mg of Betaine.

In another aspect, the composition used in the method of treating, comprises:
a. 2-12 mcg of vitamin B₁₂;
b. 100-300 mg folic acid;
c. 0.5-4 mg of vitamin B₆;
d. 200-400 mg of L-methionine; and,
e. 200-400 mg of Betaine.

In another aspect, the composition used in the method of treating, comprises:
a. 4-8 mcg of vitamin B₁₂;
b. 150-250 mg folic acid;
c. 1-3 mg of vitamin B₆;
d. 250-350 mg of L-methionine; and,
e. 250-350 mg of Betaine.

In another aspect, the composition used in the method of treating, comprises:
a. 3 mcg of vitamin B₁₂;
b. 200 mg folic acid;
c. 2 mg of vitamin B₆;
d. 300 mg of L-methionine; and,
e. 300 mg of Betaine.

In another aspect, the composition is administered once daily.

In another aspect, the composition is administered twice daily (e.g., 12 hours apart).

In another aspect, the composition is administered weekly.

In another aspect, the composition is administered twice weekly.

In another aspect, the composition is administered monthly.

In another aspect, the composition is administered for at least 1, 2, 3, to 4 weeks. Further examples of the treatment period include at least 1, 2, 3, 4, 5, 6, 7, 8, 9, 10, 11, to 12 months. Other examples of the second treatment period include at least 0.5, 1, 15, 2, 2.5, to 3 years.

In another aspect, the composition is administered while the patient is receiving nitrous oxide treatment.

The contents of all cited references are incorporated herein in their entirety.

Numerous modifications and variations of the present invention are possible considering the above teachings. It is therefore to be understood that within the scope of the appended claims, the invention may be practiced otherwise than as specifically described herein.

We claim:

1. A supplement composition, comprising:
a. a therapeutically effective amount of vitamin B₁₂ or a precursor thereof;
b. a therapeutically effective amount of folic acid or another folate precursor;
c. a therapeutically effective amount of vitamin B₆ or a precursor thereof;
d. a therapeutically effective amount of L-methionine; and,
e. a therapeutically effective amount of Betaine.

2. The supplement composition of claim 1, comprising:
a. a therapeutically effective amount of vitamin B₁₂;
b. a therapeutically effective amount of folic acid;
c. a therapeutically effective amount of vitamin B₆;
d. a therapeutically effective amount of L-methionine; and,
e. a therapeutically effective amount of Betaine.

3. A supplement composition, consisting essentially of:
a. a therapeutically effective amount of vitamin B₁₂ or a precursor thereof;
b. a therapeutically effective amount of folic acid or another folate precursor;
c. a therapeutically effective amount of vitamin B₆ or a precursor thereof;
d. a therapeutically effective amount of L-methionine; and,
e. a therapeutically effective amount of Betaine.

4. The supplement composition of claim 3, consisting essentially of:
a. a therapeutically effective amount of vitamin B₁₂;
b. a therapeutically effective amount of folic acid;
c. a therapeutically effective amount of vitamin B₆;
d. a therapeutically effective amount of L-methionine; and,
e. a therapeutically effective amount of Betaine.

5. A supplement composition, consisting of:
a. a therapeutically effective amount of vitamin B₁₂ or a precursor thereof;
b. a therapeutically effective amount of folic acid or another folate precursor;
c. a therapeutically effective amount of vitamin B₆ or a precursor thereof;
d. a therapeutically effective amount of L-methionine; and,
e. a therapeutically effective amount of Betaine.

6. The supplement composition of claim 5, consisting of:
a. a therapeutically effective amount of vitamin B₁₂;
b. a therapeutically effective amount of folic acid;
c. a therapeutically effective amount of vitamin B₆;
d. a therapeutically effective amount of L-methionine; and,
e. a therapeutically effective amount of Betaine.

7. The supplement composition of claim 1, wherein the composition, comprises:
a. 2-12 mcg of vitamin B₁₂ or a precursor thereof;
b. 100-300 mg folic acid or another folate precursor;
c. 0.5-4 mg of vitamin B₆ or a precursor thereof;
d. 200-400 mg of L-methionine; and,
e. 200-400 mg of Betaine.

8. The supplement composition of claim 1, wherein the composition, comprises:
a. 2-12 mcg of vitamin B₁₂;
b. 100-300 mg folic acid;
c. 0.5-4 mg of vitamin B₆;
d. 200-400 mg of L-methionine; and,
e. 200-400 mg of Betaine.

9. The supplement composition of claim 1, wherein the composition, comprises:
a. 4-8 mcg of vitamin B₁₂;
b. 150-250 mg folic acid;
c. 1-3 mg of vitamin B₆;
d. 250-350 mg of L-methionine; and,
e. 250-350 mg of Betaine.

10. The supplement composition of claim 1, wherein the composition, comprises:
a. 3 mcg of vitamin B₁₂;
b. 200 mg folic acid;
c. 2 mg of vitamin B₆;
d. 300 mg of L-methionine; and,
e. 300 mg of Betaine.

11. A method of treating a subject, comprising: administering a therapeutically effective amount of a composition, comprising:
a. vitamin B₁₂ or a precursor thereof;
b. folic acid or another folate precursor;
c. vitamin B₆ or a precursor thereof;
d. L-methionine; and,
e. Betaine;
wherein the subject is receiving current or chronic nitrous oxide treatment.

12. A method of treating a subject, comprising: administering a therapeutically effective amount of a composition, consisting essentially of:
a. vitamin B₁₂ or a precursor thereof;
b. folic acid or another folate precursor;
c. vitamin B₆ or a precursor thereof;
d. L-methionine; and,
e. Betaine;
wherein the subject is receiving current or chronic nitrous oxide treatment.

13. A method of treating a subject, comprising: administering a therapeutically effective amount of a composition, consisting of:
a. vitamin B₁₂ or a precursor thereof;
b. folic acid or another folate precursor;
c. vitamin B₆ or a precursor thereof;
d. L-methionine; and,
e. Betaine;
wherein the subject is receiving current or chronic nitrous oxide treatment.

14. The method of claim 11, wherein the subject who is receiving current or chronic nitrous oxide treatment is known to have at least one of the following conditions:
a. vitamin B₁₂ deficiency;
b. folate deficiency;
c. vitamin B₆ deficiency; and,
d. elevated homocysteine levels.

15. The method of claim 11, wherein the composition, comprises:
a. vitamin B₁₂;
b. folic acid;
c. vitamin B₆;
d. L-methionine; and,
e. Betaine.

16. The method of claim 11, wherein the composition, comprises:
a. 2-12 mcg of vitamin B₁₂ or a precursor thereof;
b. 100-300 mg folic acid or another folate precursor;
c. 0.5-4 mg of vitamin B₆ or a precursor thereof precursor;
d. 200-400 mg of L-methionine; and,
e. 200-400 mg of Betaine.

17. The method of claim 11, wherein the composition, comprises:
a. 2-12 mcg of vitamin B₁₂;
b. 100-300 mg folic acid;
c. 0.5-4 mg of vitamin B₆;
d. 200-400 mg of L-methionine; and,
e. 200-400 mg of Betaine.

18. The method of claim 11, wherein the composition, comprises:
a. 4-8 mcg of vitamin B₁₂;
b. 150-250 mg folic acid;
c. 1-3 mg of vitamin B₆;
d. 250-350 mg of L-methionine; and,
e. 250-350 mg of Betaine.

19. The method of claim 11, wherein the composition, comprises:
a. 3 mcg of vitamin B₁₂;
b. 200 mg folic acid;
c. 2 mg of vitamin B₆;
d. 300 mg of L-methionine; and,
e. 300 mg of Betaine.
